(19) United States
(12) Patent Application Publication
(10) Pub. No.: US 2026/0209177 A1
(43) Pub. Date: Jul. 23, 2026

(54) 1-DEOXYNOJIRIMYCIN DERIVATIVE AND USE THEREOF

(71) Applicant: ZHEJIANG UNIVERSITY, HANGZHOU, Zhejiang Province (CN)

(72) Inventors: QINGFENG YAN, HANGZHOU, ZHEJIANG PROVINCE (CN); XUE DING, HANGZHOU, ZHEJIANG PROVINCE (CN); QIANQIAN ZHUANG, HANGZHOU, ZHEJIANG PROVINCE (CN)

(21) Appl. No.: 19/139,838

(22) PCT Filed: Oct. 30, 2023
(86) PCT No.: PCT/CN2023/127606

§ 371 Date: Jun. 16, 2025

(30) Foreign Application Priority Data
Dec. 6, 2022 (CN) 202211631240.2
Aug. 7, 2023 (CN) 202310985009.1

(51) Int. Cl. C07D 211/46; A61K 31/445; A61K 31/4535; A61P 25/00; C07D 409/06

(52) U.S. Cl. CPC C07D 211/46; A61K 31/445; A61K 31/4535; A61P 25/00; C07D 409/06

(57) ABSTRACT

Disclosed are a 1-deoxynojirimycin derivative and use thereof. According to the present invention, a variety of 1-deoxynojirimycin derivatives are prepared and obtained through screening. Compared with 1-deoxynojirimycin as a lead compound, the 1-deoxynojirimycin derivative can better bind to an amino acid site that is related to a target protein OPA1, stabilize a binding pocket at a dimer interface, promote the formation of an OPA1 dimer and repair a mitochondrial ultrastructure, thereby significantly saving mitochondrial functions and effectively improving a physiological state of cells. The 1-deoxynojirimycin derivative of the present invention can be used for preparing a drug for treating a disease that is related to unbalanced formation of the OPA1 dimer, and for example, can be used as a potential therapeutic drug for mitochondrial cardiomyopathy and other mitochondrial diseases.

### FIELD OF TECHNOLOGY

The present invention relates to the technical field of biomedicine, in particular to a 1-deoxynojirimycin derivative and use thereof.

### BACKGROUND TECHNOLOGY

Mitochondria are important organelles, which are not only cellular energy factories for providing adenosine triphosphate (ATP) for the body, but also important sites for production of reactive oxygen, regulation of cell apoptosis, transduction of Ca²⁺ signals, etc. Various kinds of clinical diseases that are caused by mitochondrial dysfunction are collectively referred to as mitochondrial diseases.

Mitochondrial cardiomyopathy (MIM510000) is a type of mitochondrial disease having abnormal mitochondrial structures or functions and involving the heart and skeletal muscles, and patients often have severe heart failure. Common clinical manifestations include symptoms such as dyspnea on exertion, tachycardia, systemic myasthenia with severe systemic edema, as well as cardiomegaly and hepatomegaly, etc. The heart stays in a constant contraction and relaxation state and is an organ with highest consumption of oxygen and energy in the body, and cardiomyocytes need to rapidly and effectively supply energy by oxidation to provide sufficient ATP for the heart to maintain a normal blood pumping function. Myocardial energy metabolism is mainly completed in the mitochondria, and the ATP is synthesized through oxidative phosphorylation in the mitochondria. Therefore, the mitochondria are crucial for maintaining energy metabolism homeostasis in the cardiomyocytes and ensuring energy demands of the heart. The mitochondrial dysfunction leads to abnormal energy metabolism and oxidative stress responses and induces the occurrence and development of cardiovascular diseases, such as dilated cardiomyopathy and hypertrophic cardiomyopathy, thereby promoting the progression of heart failure. In an early stage, a mitochondrial hypertrophic cardiomyopathy (HCM)-specific induced pluripotent stem cell and directionally differentiated cardiomyocytes thereof (iPSC-CMs) are successfully constructed (Li S, et al. Mitochondrial dysfunctions contribute to hypertrophic cardiomyopathy in patient iPSC-derived cardiomyocytes with MT-RNR2 mutation. Stem Cell Reports. 2018; 10:808-821.), and the cardiomyocytes have a pathological phenotype similar to that of cardiomyocytes in patients with HCM, showing obvious mitochondrial dysfunction.

Maintaining mitochondrial homeostasis is crucial for regulating cardiac energy metabolism. Therefore, targeting the mitochondria to improve their functions is of great significance for the prevention and treatment of cardiovascular diseases. Currently, conventional drugs for treating cardiomyopathy include β-receptor blockers, Ca²⁺ channel blockers, antiarrhythmic drugs, calcium desensitizers and metabolism and contraction regulators, etc. However, most of these drugs are inhibitors and focus on single types, which especially have poor applicability and unsatisfactory therapeutic effects for mitochondrial cardiomyopathy. The mitochondria are one of the important targets for treating the cardiomyopathy. However, most of current mitochondrial drugs are for symptomatic treatment with single improvement approaches, such as regulating biogenesis of the mitochondria in cells, an NAD⁺ level and a reactive oxygen level of the mitochondria and inducing mitochondrial metabolic reprogramming, which cannot fundamentally improve overall functions of the mitochondria. Therefore, it is of great significance to research and develop innovative drugs capable of directly targeting the mitochondria, reconstructing a mitochondrial ultrastructure and reshaping mitochondria functions, thereby bringing new hope for treating the mitochondrial cardiomyopathy and other mitochondrial diseases.

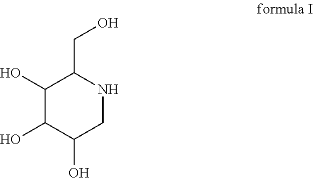 [SMILES: OCC1NCC(O)C(O)C1O]

1-deoxynojirimycin (DNJ) is a polyhydroxy alkaloid (as shown in formula I), which is mainly used as an α-glycosidase inhibitor for regulating glycometabolism and also has functions such as reducing blood glucose, lowering lipid, resisting tumors, resisting viruses, etc. In an early stage, by using specific transmitochondrial cells (cybrids) of patients with the mitochondrial cardiomyopathy and the iPSC-cardiomyocytes, it has found through a two-step screening strategy that the compound DNJ can target a mitochondrial protein OPA1 and improve the mitochondrial ultrastructure of patients with the mitochondrial cardiomyopathy by promoting the formation of an OPA1 dimer, thereby improving functions of the mitochondria (patent applied, under substantive examination). However, the DNJ has the following shortcomings in extraction and use: (1) the DNJ has a low content in natural products and a low extraction rate, thereby increasing the production cost of DNJ; (2) chemical synthesis methods have complex processes, long cycles, low recovery rates and environmental pollution, etc.; and (3) due to low bioavailability and poor lipophilicity, the DNJ with effective and long-lasting activity cannot be maintained in vivo. Therefore, how to improve the lipophilicity of DNJ, increase the bioavailability, enhance the affinity to tissues and improve the targetability to develop novel DNJ derivatives is an effective strategy to promote the transformation into clinical practical applications.

Traditional DNJ derivatives, based on differences in modification sites, are mainly classified into the following three categories: N-substituted derivatives, C-substituted derivatives and O-substituted derivatives, wherein the N-substituted derivatives are the most conventional and classic, such as miglitol and miglustat. Both the two derivatives can be widely used as a strong α-glucosidase inhibitor for treating type II diabetes. Particularly, the miglustat, approved for marketing in Europe in 2003, is capable of passing through a blood-brain barrier to prevent the synthesis of a glycolipid, thereby providing a new solution for treating various lysosomal storage diseases that are related to the central nervous system. Although DNJ derivatives with better performance have been constantly developed, the derivatives are still limited to conventional known pharmacological effects, such as resisting diabetes, resisting tumors, resisting viruses, etc.

Optic atrophy protein-1 (OPA1) is a dynamin that is mainly located on a mitochondrial inner membrane, and an OPA1 multimer is crucial for maintaining mitochondrial morphology. The OPA1 with functional defects leads to mitochondrial cristae disorder, mitochondrial fission, etc., thereby causing a series of diseases, such as optic atrophy, progressive external ophthalmoplegia and ataxia, progressive myoclonic epilepsy, spastic paresis and intestinal motility disorder, and retinal degeneration. Currently, there are few drugs with the OPA1 as a target, and most of the drugs are inhibitors. It has been reported that MYLS22 achieves the effect of inhibiting tumor growth by inhibiting the expression of OPA1.

Currently, there are no novel DNJ derivatives that target the OPA1 and are used as agonists to improve the mitochondrial ultrastructure and biological functions for treating mitochondrial diseases, such as mitochondrial cardiomyopathy, epicophosis, optic atrophy, progressive external ophthalmoplegia and ataxia, progressive myoclonic epilepsy, spastic paresis and intestinal motility disorder, and retinal degeneration. In the present application, an innovative DNJ derivative and use thereof are provided.

### SUMMARY OF THE INVENTION

In view of the shortcomings in the prior art, the present invention provides a 1-deoxynojirimycin derivative and use thereof. Compared with 1-deoxynojirimycin as a lead compound, the derivative has better activity to bind to a target protein OPA1.

A 1-deoxynojirimycin derivative is one of the following:
(1) DNJ-1, having a structure of formula II:

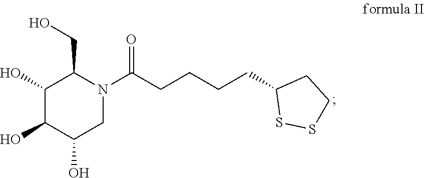 [SMILES: O=C(CCCC[C@@H]1CCSS1)N1C[C@H](O)[C@@H](O)[C@H](O)[C@H]1CO ; ";"]

(2) DNJ-5a, having a structure of formula VII:

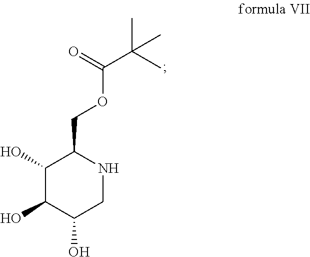 [SMILES: CC(C)(C)C(=O)OC[C@H]1NC[C@H](O)[C@@H](O)[C@@H]1O ; ";"]

(3) DNJ-5c, having a structure of formula VIII:

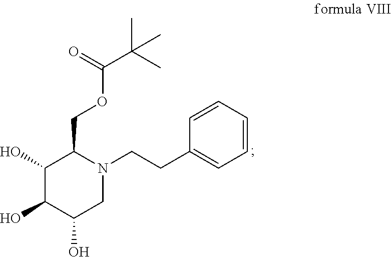 [SMILES: CC(C)(C)C(=O)OC[C@@H]1[C@@H](O)[C@H](O)[C@@H](O)CN1CCc1ccccc1 ; ";"]

and
(4) having a structure of general formula I-I:

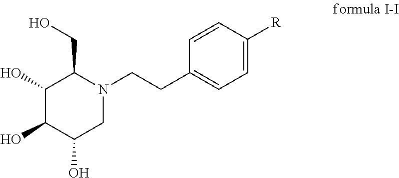

wherein R is selected from —H, —NO₂, and —X, wherein X is F, Cl, Br, or I.

Preferably, R is selected as —X, and X is F.

When R is selected as —H, the derivative is DNJ-3a, having a structure of formula III:

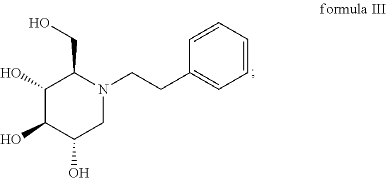 [SMILES: OC[C@@H]1[C@@H](O)[C@H](O)[C@@H](O)CN1CCc1ccccc1 ; ";"]

when R is selected as —F, the derivative is DNJ-3b, having a structure of formula IV:

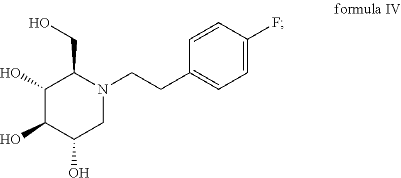 [SMILES: OC[C@@H]1[C@@H](O)[C@H](O)[C@@H](O)CN1CCc1ccc(F)cc1]

and
when R is selected as —NO₂, the derivative is DNJ-3c, having a structure of formula V:

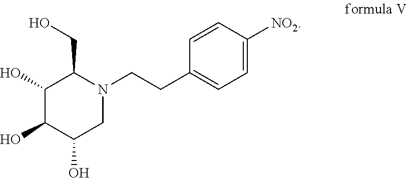 [SMILES: O=[N+]([O-])c1ccc(CCN2C[C@H](O)[C@@H](O)[C@H](O)[C@H]2CO)cc1]

The present invention further provides use of the 1-deoxynojirimycin derivative or a pharmaceutically acceptable salt thereof in preparation of an OPA1 agonist, wherein the 1-deoxynojirimycin derivative or a pharmaceutically acceptable salt thereof is capable of promoting polymerization of OPA1 from a monomer to a multimer.

The present invention further provides use of the 1-deoxynojirimycin derivative or a pharmaceutically acceptable salt thereof in preparation of a drug for treating a disease that is related to unbalanced formation of an OPA1 multimer. Preferably, the disease that is related to unbalanced formation of the OPA1 multimer is hypertrophic cardiomyopathy, epicophosis, optic atrophy, progressive external ophthalmoplegia and ataxia, progressive myoclonic epilepsy, spastic paresis and intestinal motility disorder, or retinal degeneration. More preferably, the disease that is related to unbalanced formation of the OPA1 multimer is caused by mitochondrial dysfunction.

The present invention further provides a drug for treating a disease that is related to unbalanced formation of an OPA1 multimer, wherein an effective ingredient is the 1-deoxynojirimycin derivative or a pharmaceutically acceptable salt thereof. Preferably, the disease that is related to unbalanced formation of the OPA1 multimer is hypertrophic cardiomyopathy, epicophosis, optic atrophy, progressive external ophthalmoplegia and ataxia, progressive myoclonic epilepsy, spastic paresis and intestinal motility disorder, or retinal degeneration. More preferably, the disease that is related to unbalanced formation of the OPA1 multimer is caused by mitochondrial dysfunction.

The present invention further provides a method for preparing the 1-deoxynojirimycin derivative. When the 1-deoxynojirimycin derivative is the above (1), compounds a and b are taken and allowed to undergo a condensation reaction in a solvent to obtain the corresponding 1-deoxynojirimycin derivative, wherein the compound a is 1-deoxynojirimycin, and the compound b is:

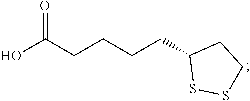 [SMILES: O=C(O)CCCC[C@@H]1CCSS1 ; ";"]

when the 1-deoxynojirimycin derivative is the above (2), a nitrogen site in a nitrohexyl ring of 1-deoxynojirimycin needs to be protected first, then a condensation reaction is carried out at a hydroxymethyl site, thus, the 1-deoxynojirimycin first reacts with benzyl chloroformate to generate an intermediate product 1:

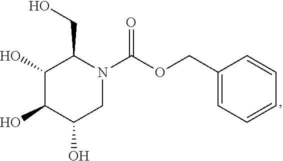 [SMILES: O=C(OCc1ccccc1)N1C[C@H](O)[C@@H](O)[C@H](O)[C@H]1CO ; ","]

then, the intermediate product 1 reacts with PivCl to obtain an intermediate product 2:

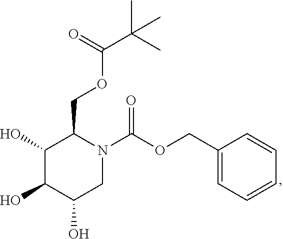 [SMILES: CC(C)(C)C(=O)OC[C@@H]1[C@@H](O)[C@H](O)[C@@H](O)CN1C(=O)OCc1ccccc1 ; ","]

and finally, the intermediate product 2 is reduced to obtain the 1-deoxynojirimycin derivative, wherein during reduction, the intermediate product 2 is first dissolved in MeOH, and then Pd/C is added to carry out a reaction under the protection of H₂; when the 1-deoxynojirimycin derivative is the (3), 1-deoxynojirimycin first reacts with 2-bromoethylbenzene to generate an intermediate product:

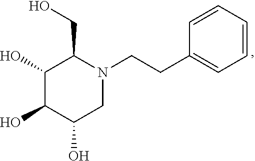 [SMILES: OC[C@@H]1[C@@H](O)[C@H](O)[C@@H](O)CN1CCc1ccccc1 ; ","]

and then, the intermediate product reacts with PivCl to obtain the 1-deoxynojirimycin derivative; and
when the 1-deoxynojirimycin derivative is the above (4), compounds a and b4 are taken and allowed to undergo a condensation reaction in a solvent to obtain the corresponding 1-deoxynojirimycin derivative, wherein the compound a is 1-deoxynojirimycin, and the compound b4 is:

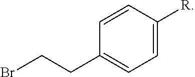

According to the present invention, a variety of 1-deoxynojirimycin derivatives are prepared and obtained through screening. Compared with the lead compound 1-deoxynojirimycin, the 1-deoxynojirimycin derivative can better bind to an amino acid site that is related to a target protein OPA1, stabilize a binding pocket at a dimer interface, promote the formation of an OPA1 dimer and repair a mitochondrial ultrastructure, thereby significantly saving mitochondrial functions and effectively improving a physiological state of cells. The 1-deoxynojirimycin derivative of the present invention can be used for preparing a drug for treating a disease that is related to unbalanced formation of the OPA1 dimer, and for example, can be used as a potential therapeutic drug for mitochondrial cardiomyopathy and other mitochondrial diseases.

### BRIEF DESCRIPTION OF THE DRAWINGS

FIG. 1 shows a reaction equation of structural formula II.

FIG. 2 shows a mass spectrometry analysis diagram of DNJ-1.

FIG. 3 shows a reaction equation of structural formula III.

FIG. 4 shows a nuclear magnetic resonance (NMR) spectrum of DNJ-3a.

FIG. 5 shows a reaction equation of structural formula IV.

FIG. 6 shows an NMR spectrum of DNJ-3b.

FIG. 7 shows a reaction equation of structural formula V.

FIG. 8 shows an NMR spectrum of DNJ-3c.

FIG. 9 shows a reaction equation of structural formula VI.

FIG. 10 shows an NMR spectrum of DNJ-4d.

FIG. 11 shows a reaction equation of structural formula VII.

FIG. 12 shows an NMR spectrum of DNJ-5a.

FIG. 13 shows a reaction equation of structural formula VIII.

FIG. 14 shows a mass spectrometry analysis diagram of DNJ-5c.

FIG. 15 shows immunofluorescence detection results of cardiomyocytes differentiated from wild-type and mitochondrial HCM-specific iPSCs.

FIG. 16 shows preliminary screening results of different DNJ derivatives on the mitochondrial membrane potential of mitochondrial HCM-specific Cybrids.

FIG. 17 shows polyclonal secondary screening results of different DNJ derivatives on the mitochondrial membrane potential of mitochondrial HCM-specific Cybrids, wherein A shows enhancement effects of the DNJ derivatives on the mitochondrial membrane potential; and B shows impacts of the derivatives DNJ-1 and DNJ-5a on the mitochondrial membrane potential of a control group (Con) and HCM Cybrids. n=9, and ***P<0.001.

FIG. 18 shows impact detection results of some derivatives on the mitochondrial activity of mitochondrial HCM-specific iPSC-CMs compared with lead compound DNJ, wherein A shows the survival rate of cells in a galactose culture medium at different days; and B shows the survival rate of cells on the third day after drug administration. n=3, and ###P<0.001, indicating comparison with a Con-CMs group. *P<0.05, **P<0.01, and ***P<0.001, indicating comparison with an HCM-CMs group.

FIG. 19 shows detection results of half effective concentrations of derivatives DNJ-1 and DNJ-5a in HCM iPSC-CMs compared with DNJ, wherein A shows the half effective concentration of DNJ; B shows the half effective concentration of DNJ-1; and C shows the half effective concentration of DNJ-5a.

FIG. 20 shows detection results of the affinity between derivatives DNJ-1 and DNJ-5a and a purified OPA1-EGFP protein compared with DNJ, wherein A shows Coomassie brilliant blue identification results of the purified protein; B shows an affinity curve of DNJ; C shows an affinity curve of DNJ-1; and D shows an affinity curve of DNJ-5a.

FIG. 21 shows recovery effects of derivatives DNJ-1 and DNJ-5a on the action potential of iPSC-CMs, wherein A shows electrophysiological conditions of statistically abnormal cardiomyocytes; and B shows electrophysiological representatives of cardiomyocytes treated with DNJ-5a.

FIG. 22 shows cardiac uhrasonography results of mice with Ang II-induced myocardial hypertrophy after treatment with a derivative DNJ-5a compared with DNJ, wherein A shows representatives of the cardiac uhrasonography results of the mice; and B-E show statistical results of relevant parameters of cardiac uhrasonography of the mice. In Sham, n=8. In Sham+DNJ-5a, n=7. In AngII, n=8. In AngII+DNJ, n=9. In AngII+DNJ-5a, n=9. *P<0.05, **P<0.01, and ***P<0.001.

FIG. 23 shows results of the size and weight of hearts of mice, wherein A shows hematoxylin-eosin (HE) staining representatives of maximum cross sections of the hearts of the mice in different groups; and B shows statistical results of the heart weight (mg)/body weight (g) of the mice. In Sham, n=8. In Sham+DNJ-5a, n=7. In AngII, n=8. In AngII+DNJ, n=9. In AngII+DNJ-5a, n=9. *P<0.05, **P<0.01, and ***P<0.001.

FIG. 24 shows typical representatives of mitochondrial transmission electron microscopy images of heart tissues of mice.

### DETAILED DESCRIPTION OF THE EMBODIMENTS

Structural optimization of DNJ is mainly considered in the following directions: an aryl group or an aralkyl group is introduced to improve the lipid solubility, bioavailability and activity of compounds; and an amino acid is introduced to improve the affinity to tissues and improve the targetability, etc.

According to structures of the DNJ and natural products capable of improving the mitochondrial membrane potential, structural modification of the DNJ is mainly performed by adopting the following several strategies:
(1) based on a combination principle, structural fragments of compounds capable of improving the mitochondrial membrane potential, such as salidroside and lipoic acid, are selected and combined with the DNJ to investigate impacts of target compounds on activity;
(2) an aryl group or an aralkyl group is introduced onto a nitrogen atom to improve the lipid solubility, bioavailability and activity of compounds;
(3) an amino acid is introduced onto a nitrogen atom to improve the affinity to tissues and improve the targetability; and
(4) a quaternary ammonium cation is introduced to improve the affinity of compounds to a mitochondrial membrane and improve the mitochondrial targetability.

During practical synthesis, a total of 10 compounds in four series are obtained, and the compounds with the structural optimization of DNJ obtained in practical synthesis all have a purity of above 90% after purification.

### Example 1

(1) A compound name is DNJ-1; a molecular formula is C₁₄H₂₅NO₅S₂; a structural name is 5-((R)-1,2-dithiolan-3-yl)-1-((2R,3R,4R,5S)-3,4,5-trihydroxy-2-(hydroxymethyl) piperidin-1-yl) p entan-1-one; and a structure is shown as formula II:

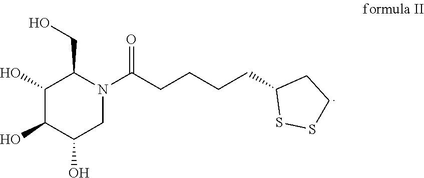 [SMILES: O=C(CCCC[C@@H]1CCSS1)N1C[C@H](O)[C@@H](O)[C@H](O)[C@H]1CO ; "."]

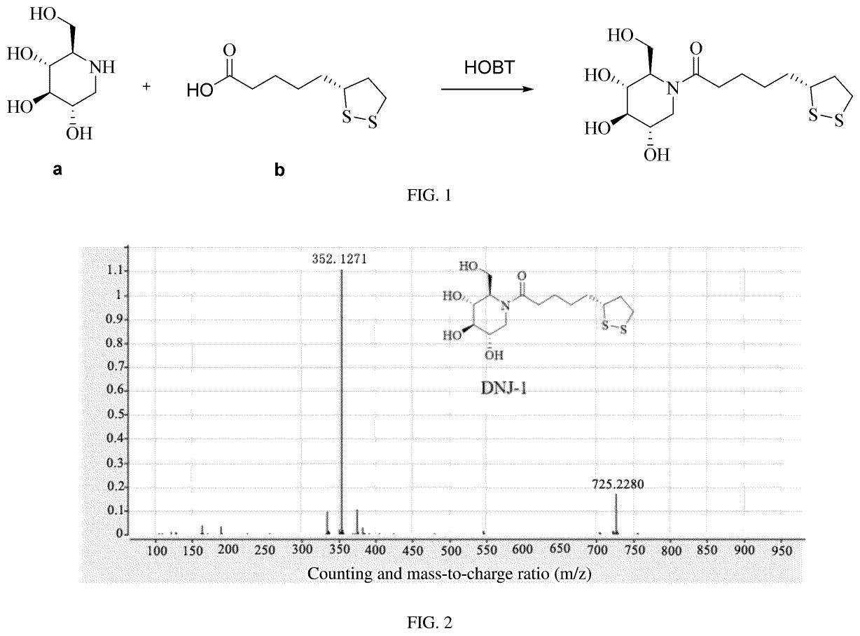

The reaction equation is shown in FIG. 1.

Compounds a (193.97 mg, 1.19 mmol) and b (245 mg, 1.19 mmol) were taken into a round-bottom flask, dichloromethane (DCM, 10 ml) was added to as a solvent, the temperature was lowered to 0° C., HOBT (0.177 g, 1.31 mmol) and EDCI (0.251 g, 1.31 mmol) were added, and then, a reaction was carried out at room temperature for 10 hours. After the reaction was completed, the solvent was removed by evaporation, and separation was performed by column chromatography to obtain a product.

FIG. 2 shows a mass spectrometry analysis diagram of the DNJ-1.

(2) A compound name is DNJ-3a; a molecular formula is C₁₄H₂₁NO₄; a structural name is (2R,3R,4R,5S)-2-(hydroxymethyl)-1-phenethylpiperidine-3,4,5-triol; and a structure is shown as formula III:

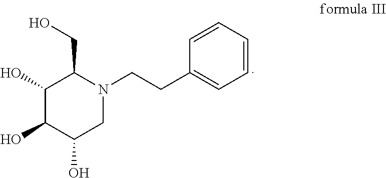 [SMILES: OC[C@@H]1[C@@H](O)[C@H](O)[C@@H](O)CN1CCc1ccccc1 ; "."]

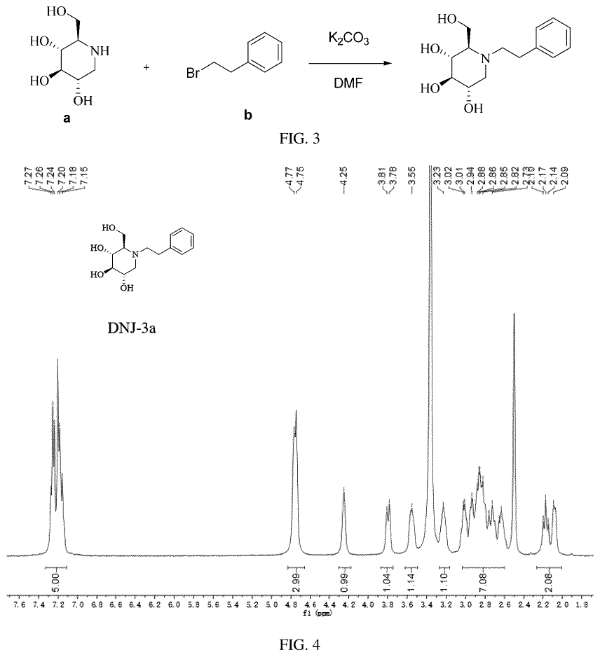

A reaction equation is shown in FIG. 3.

Compound a (163 mg, 1 mmol), compound b (220.79 mg, 1.2 mmol) and potassium carbonate (414 mg, 3 mmol) were taken into a sealed tube, dimethylformamide (DMF) (5 mL) was added to serve as a solvent, and heating was performed to carry out a reaction at 80° C. for 4 hours. The reaction was completed according to monitoring by thin-layer chromatography (TLC). The solvent was removed by evaporation, and column chromatography was performed to obtain about 80 mg of a product.

FIG. 4 shows an NMR spectrum of the DNJ-3a.

(3) A compound name is DNJ-3b; a molecular formula is C₁₄H₂₀FNO₄; a structural name is (2R,3R,4R,5S)-1-(4-fluorophenethyl)-2-(hydroxymethyl) piperidine-3,4,5-triol; and a structure is shown as formula IV:

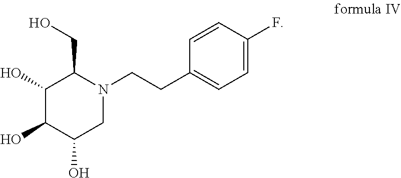 [SMILES: OC[C@@H]1[C@@H](O)[C@H](O)[C@@H](O)CN1CCc1ccc(F)cc1]

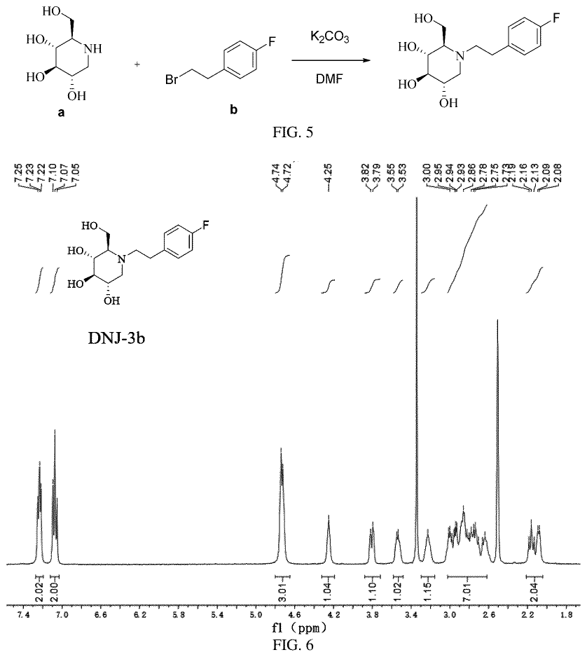

A reaction equation is shown in FIG. 5.

Compound a (163 mg, 1 mmol), compound b (242.38 mg, 1.2 mmol) and potassium carbonate (414 mg, 3 mmol) were weighed and taken into a sealed tube, DMF was used as a solvent, and heating was performed to carry out a reaction at 80° C. for 4 hours. The reaction was completed according to monitoring by a thin-layer chromatography plate, the solvent was removed by rotary evaporation, and column chromatography was performed to obtain about 70 mg of a product.

FIG. 6 shows an NMR spectrum of the DNJ-3b.

(4) A compound name is DNJ-3c; a molecular formula is C₁₄H₂₀N₂O₆; a structural name is (2R,3R,4R,5S)-2-(hydroxymethyl)-1-(4-nitrophenethyl) piperidine-3,4,5-triol; and a structure is shown as formula V:

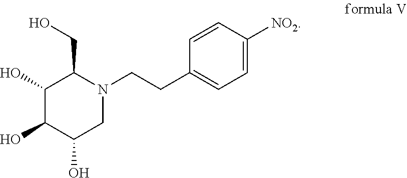 [SMILES: O=[N+]([O-])c1ccc(CCN2C[C@H](O)[C@@H](O)[C@H](O)[C@H]2CO)cc1]

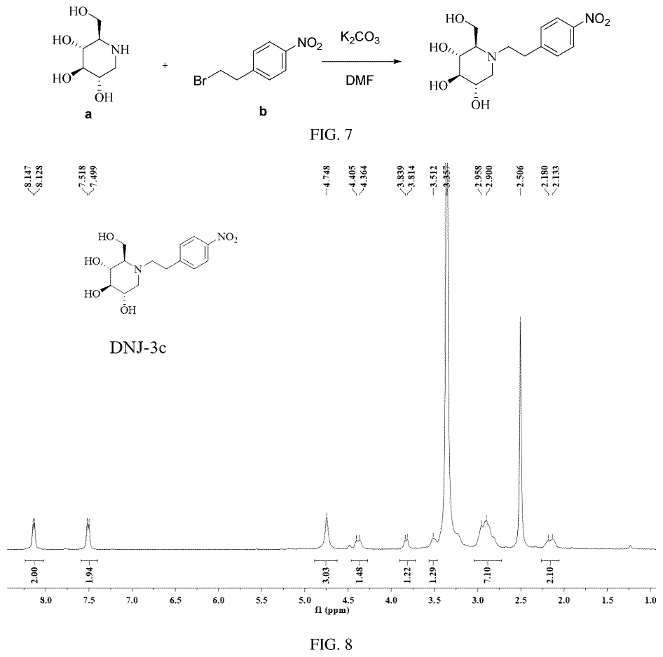

A reaction equation is shown in FIG. 7.

Compound a (163 mg, 1 mmol), compound b (274.8 mg, 1.2 mmol) and potassium carbonate (414 mg, 3 mmol) were weighed and taken into a sealed tube, DMF was used as a solvent, and heating was performed to carry out a reaction at 80° C. for 4 hours. The reaction was completed according to monitoring by a thin-layer chromatography plate, the solvent was removed by rotary evaporation, and column chromatography was performed to obtain about 120 mg of a product.

FIG. 8 shows an NMR spectrum of the DNJ-3c.

(5) A compound name is DNJ-4d; a molecular formula is C₁₅H₁₈N₂O₅; a structural name is 4-(2-((2R,3R,4R,5S)-3,4,5-trihydroxy-2-(hydroxymethyl) piperidin-1-yl) acetyl)benzonitrile; and a structure is shown as formula VI:

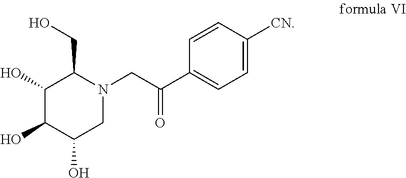 [SMILES: N#Cc1ccc(C(=O)CN2C[C@H](O)[C@@H](O)[C@H](O)[C@H]2CO)cc1]

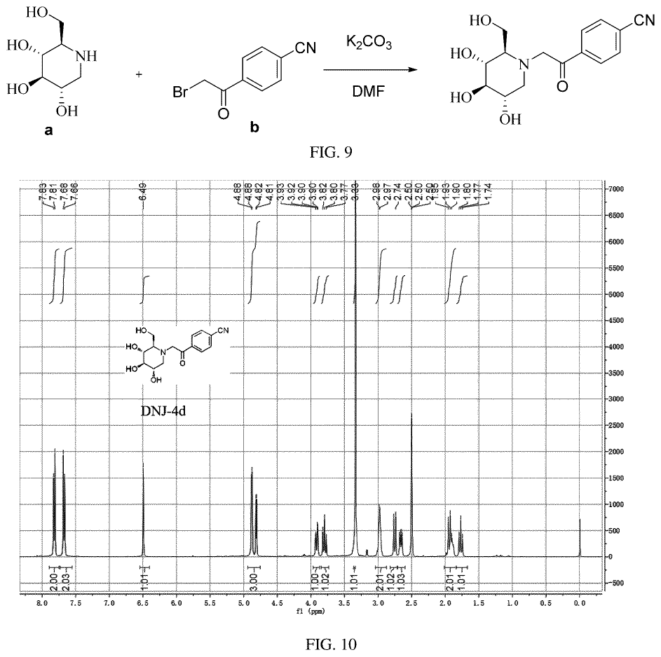

A reaction equation is shown in FIG. 9.

Compound a (163 mg, 1 mmol), compound b (267.6 mg, 1.2 mmol) and potassium carbonate (414 mg, 3 mmol) were weighed and taken into a sealed tube, DMF was used as a solvent, and heating was performed to carry out a reaction at 80° C. for 4 hours. The reaction was completed according to monitoring by a thin-layer chromatography plate, the solvent was removed by rotary evaporation, and column chromatography was performed to obtain about 90 mg of a product.

FIG. 10 shows an NMR spectrum of the DNJ-4d.

(6) A compound name is DNJ-5a; a molecular formula is C₁₁H₂₁NO₅; a structural name is ((2R,3R,4R,5S)-3,4,5-trihydroxypiperidin-2-yl)methyl pivalate; and a structure is shown as formula VII:

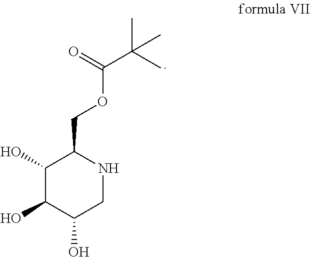 [SMILES: CC(C)(C)C(=O)OC[C@H]1NC[C@H](O)[C@@H](O)[C@@H]1O ; "."]

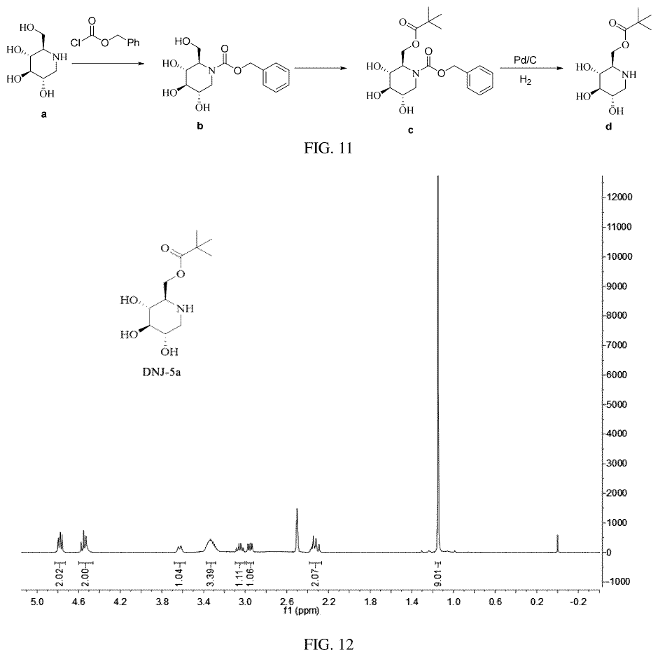

A reaction equation is shown in FIG. 11.

Compound a (115 mg, 0.7 mmol) was weighed and dissolved in a mixture of dioxane and water (1:1, 12 mL), and then, sodium chloride (1.75 equivalents) and benzyl chloroformate (1.54 equivalents) were added and stirred at room temperature for 18 hours. Then, the dioxane was removed by rotary evaporation, extraction was performed with dichloromethane, and an organic layer was retained. Column chromatography was performed to obtain product b, the compound b (149 mg, 0.5 mmol) was dissolved in pyridine (3 mL), and PivCl (2 times equivalents) was slowly added at 0° C. PivCl (2 times equivalents) was further added after 1 hour, and the reaction mixture was diluted with ethyl acetate and methanol after 3 hours. The solvent was removed under reduced pressure, and column chromatography was performed to obtain compound c.

The compound c (150 mg, 0.39 mmol) was dissolved in MeOH (5 mL), and then, 10% Pd/C (30 mg) was added. The mixture was stirred at room temperature and protected with H₂ overnight. Column chromatography was performed to obtain compound d.

FIG. 12 shows an NMR spectrum of the DNJ-5a.

(7) A compound name is DNJ-5c; a molecular formula is C₁₉H₂₉NO₅; a structural name is ((2R,3R,4R,5S)-3,4,5-trihydroxy-1-phenethylpiperidin-2-yl)methyl pivalate; and a structure is shown as formula VIII:

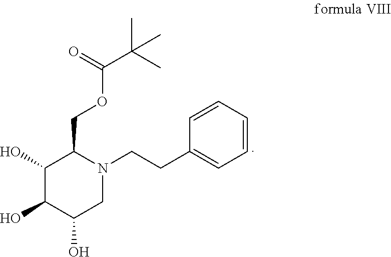 [SMILES: CC(C)(C)C(=O)OC[C@@H]1[C@@H](O)[C@H](O)[C@@H](O)CN1CCc1ccccc1 ; "."]

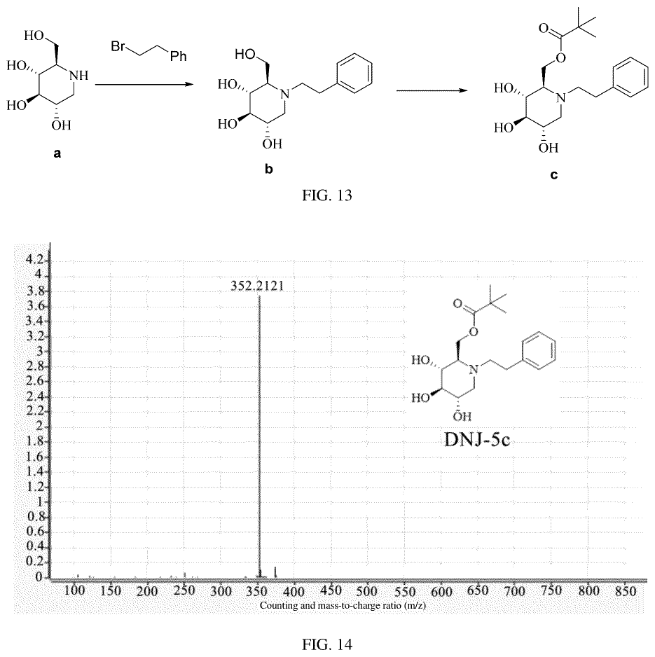

A reaction equation is shown in FIG. 13.

Compound a (163 mg, 1 mmol), 2-bromoethylbenzene (220.79 mg, 1.2 mmol) and potassium carbonate (414 mg, 3 mmol) were weighed and taken into a sealed tube, DMF was used as a solvent, and heating was performed to carry out a reaction at 80° C. for 4 hours. The reaction was completed according to monitoring by a TLC plate, the solvent was removed by rotary evaporation, and column chromatography was performed to obtain about 80 mg of an intermediate product (compound b).

The compound b (133.5 mg, 0.5 mmol) was dissolved in pyridine (3 mL), and PivCl (2 times equivalents) was slowly added at 0° C. PivCl (2 times equivalents) was further added after 1 hour, and the reaction mixture was diluted with ethyl acetate and methanol after 3 hours. The solvent was removed under reduced pressure, and column chromatography was performed to obtain compound c.

FIG. 14 shows a mass spectrometry analysis diagram of the DNJ-5c.

### Example 2

Cytosomes that were isolated by enucleation from immoralized lymphocytes of normal persons and patients with MT-RNR2 mutation were fused with ρ0 cells lacking mitochondrial DNA to construct mitochondrial-specific transmitochondrial cells (Cybrids) with a same nuclear background (referring to a previously published paper in a laboratory for a specific method: Li D, Sun Y, et al. Mitochondrial dysfunction caused by m.2336T>C mutation with hypertrophic cardiomyopathy in cybrid cell lines. Mitochondrion. 2019 May; 46:313-320.). The cell line was previously constructed and preserved in the laboratory.

Urine cells of patients with MT-RNR2 mutation and hypertrophic cardiomyopathy were used to induce iPSCs and differentiate the same into iPSC-CMs (referring to a previously published paper in a laboratory for a specific method: Li S, Pan H, Tan C, et al. Mitochondrial dysfunctions contribute to hypertrophic cardiomyopathy in patient iPSC-derived cardiomyocytes with MT-RNR2 mutation. Stem Cell Reports. 2018; 10:808-821.). In brief, in 3-4 days before induced differentiation, iPSCs were digested into single cells with Accutase (Stem cell) and resuspended in an mTeSR1 (Stem cell) culture solution, and 10⁵ cells were evenly inoculated onto a six-well plate with Matrigel (BD) as a base. On day 0, when a cell density reached about 95%, the culture solution was changed into RPMI/B27-insulin (Gibco, Cat. no. A1895601) and 12 μM CHIR99021 (selleck, Cat. no. CT99021) for culture for 24 hours. On day 1, the CHIR99021 in the cells was removed and changed into an RPMI/B27-insulin culture solution for continued culture. On days 2-3, 5 μmol/L IWP2 (Tocris, Cat. no. 3533) was given and dissolved in the RPMI/B27-insulin to treat the cells for 2 days. On days 3-4, the IWP2 in the cells was removed. After continued culture with the RPMI/B27-insulin culture solution for 2 days, the culture solution was changed into RPMI/B27 (Gibco). On days 7-12, spontaneously pulsating cardiomyocytes were observed successively.

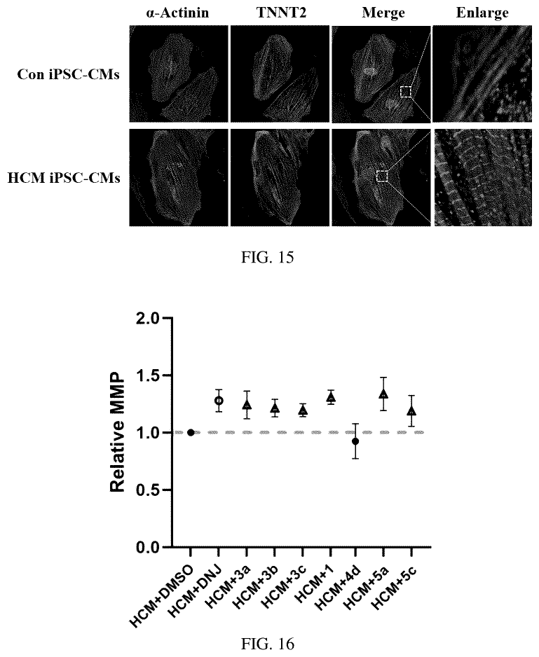

Slides were laid in 24 wells, and the cells were inoculated. After washing with PBS for three times, 4% paraformaldehyde was added for fixation at room temperature for 15 min. Then, 0.2% Triton-X100 was added for ventilation at room temperature for 15 min. Then, 3% BSA was added for sealing at room temperature for 1 hour. Then, anti-a-ACTIN (Sigma, 1:200) and TNNT2 (Abcam, 1:200) were added overnight at 4° C. Then, fluorescent secondary antibodies (the secondary antibodies were Goat Anti-Rabbit IgG H&L (Alexa Fluor® 488) (Abcam) and Goat Anti-Mouse IgG H &L (Alexa Fluor® 594) (Abcam)) were added. The cells were incubated at room temperature in the dark for 1 hour. Further, 0.5 mL of 1 μg/mL DAPI was added and placed at room temperature in the dark for 5 min, and the slides were sealed with 50% glycerol. During the period, washing with PBS for 3 times was required in each step. Finally, results were observed under a confocal microscope. The results are shown in FIG. 15. The immunofluorescence results show that all of the induced cardiomyocytes can successfully express the myocardial marker proteins anti-a-ACTIN and TNNT2, indicating that the HCM-iPSCs with MT-RNR2 mutation can be successfully differentiated into the cardiomyocytes through the above steps.

The combination of the patient-specific transmitochondrial cells (cybrids) and the cells directionally differentiated from iPSCs to jointly screen a mitochondrial drug is a beneficial attempt. The transmitochondrial cells eliminate impacts of nuclear genes and intuitively reflect an impact of the mitochondrial gene mutation on mitochondrial functions, and iPSC-CMs, as a more powerful model, provide more physiological results for more accurate drug evaluation. A two-step screening method combining the transmitochondrial cells and the iPSC-CMs creates conditions for conducting research on mechanisms of mitochondrial rescue and cardiomyocyte function recovery, which can promote the research progress of mitochondrial targeted drugs for mitochondrial diseases.

### Example 3

Transmitochondrial cells with MT-RNR2 mutation were implemented with different DNJ derivative treatments, and mitochondrial membrane potential situations were determined. In the experiment, a JC-10 mitochondrial membrane potential assay kit (Abcam, ab112134) was used for detection.

JC-10 has an ability to selectively enter mitochondria and is reversibly changed from green to red in color with increase of the membrane potential. In normal cells, the JC-10 is concentrated in a mitochondrial matrix to form red fluorescent aggregates. However, in apoptotic and necrotic cells, the JC-10 is diffused out of the mitochondria and changed into a monomer form to stain the cells into green fluorescent cells. Fluorescence values corresponding to Ex/Em=490 nm/525 nm and 540 nm/590 nm that are detected by a microplate reader can reflect the mitochondrial membrane potential level, and a lower ratio indicates that the mitochondria are damaged more severely.

The transmitochondrial cells were inoculated into a transparent bottom of a 96-well black plate at 0.8×10⁴ piece/well. 3 duplicate wells were set for each group. At 16 hours after the inoculation, a fresh complete culture medium (DMEM+10% FBS) was replaced. Different DNJ derivatives at 30 μM were added into an HCM-Cybrids experimental group, and DMSO of an equal volume was added into another HCM-Cybrids to serve as a control group. The cells were placed in an environment with 5% of CO₂ for culture at 37° C. for 24 hours. After 24 hours, 100×JC-10 was diluted with Assay buffer A. A diluted liquid was added into the well plate at 25 μl per well. The cells were placed in an environment with 5% of CO₂ for culture at 37° C. for 30 min. Before loading on a machine, JC-10 Buffer B was added into the well plate at 25 μL per well. Fluorescence values corresponding to Ex/Em-490 nm/525 nm and 540 nm/590 nm were detected by a microplate reader. After a corresponding blank control group was subtracted for each well, PL590/PL525 was compared.

Results are shown in FIG. 16, wherein HCM+DMSO shows the control group with addition of DMSO, HCM+DNJ shows the addition of lead compound DNJ, HCM+3a shows the addition of the DNJ derivative DNJ-3a, and the other groups also similarly show the addition of different DNJ derivatives. The results show that the derivatives DNJ-4a, 4b, 4c and 4d have poor enhancement effects on the mitochondrial membrane potential of HCM-Cybrids with MT-RNR2 mutation, and DNJ-3a, 3b, 3c, 1, 5a and 5c have significant enhancement effects on the mitochondrial membrane potential and can be used in a second round of secondary screening.

### Example 4

Polyclonal secondary screening results of different DNJ derivatives on the mitochondrial membrane potential of MT-RNR2 mutant HCM-Cybrids.

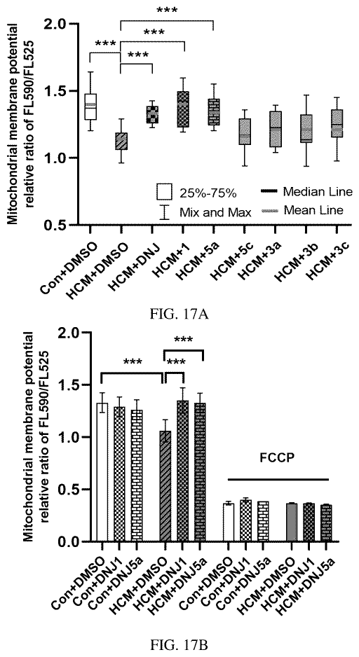

A method for detecting the mitochondrial membrane potential was the same as that previously described in Example 3. A difference is that 3 HCM-Cybrids cell clones were applied herein for detection to achieve the purpose of making the results more reliable. As shown in FIG. 17A, DNJ-1 and DNJ-5a can significantly enhance the mitochondrial membrane potential, and meanwhile, DNJ-3a, DNJ-3b and DNJ-3c, although being not significant, also have certain degrees of enhancement effects. Next, the focus was on DNJ-1 and DNJ-5a to improve the situations of the membrane potential of Cybrids cells in a normal group and the addition of FCCP in different groups, as shown in FIG. 17B.

An operation method in FIG. 17B is as follows. 30 μM DNJ-1 and DNJ-5a drugs were added into Con-Cybrids and HCM-Cybrids cells, and after 24 hours, 50 μL of 10 μM FCCP diluted with a culture medium was added into 3 duplicate wells in each group. The FCCP, as a decoupling agent, was capable of disrupting the formation of mitochondrial membrane potential. 50 μL of a culture medium was added into 3 cloned duplicate wells in each group. The cells were placed in an environment with 5% of CO₂ for culture at 37° C. for 30 min. 100×compound A (JC-10) was diluted with compound B (JC-10 Buffer A). A diluted liquid was added into a well plate at 25 μl per well. The cells were placed in an environment with 5% of CO₂ for culture at 37° C. for 30 min. Before loading on a machine, JC-10 Buffer B was added into the well plate at 25 μL per well. Fluorescence values corresponding to Ex/Em=490 nm/525 nm and 540 nm/590 nm were detected by a microplate reader. After a corresponding blank control group was subtracted for each well, PL590/PL525 was compared.

The results show that the derivatives DNJ-3a, DNJ-3b and DNJ-3c have certain degrees of enhancement effects on the mitochondrial membrane potential of HCM-Cybrids, meanwhile, DNJ-1 and DNJ-5a have most significant enhancement effects, and the two derivatives have no impact on the mitochondrial membrane potential of Con-Cybrids.

### Example 5

HCM-iPSC-CMs with MT-RNR2 mutation were implemented with different DNJ derivatives and DNJ treatments, and impacts of the derivatives on cell viability were detected through a galactose-induced cell death experiment.

The cells were inoculated into a 24-well plate at 2×10⁴ piece/well, cultured with an L-15 culture medium (Gibco) and a B27 cell culture additive, and placed in an incubator with 5% of CO₂ at 37° C. After 16 hours, the culture medium for the cells in an HCM-CMs group was replaced with an L-15 culture medium added with a drug (at a final concentration of 30 μmol/L), meanwhile, Con-CMs and HCM-CMs that were treated with an L-15 culture medium added with DMSO of a same volume were set as controls, and 3 duplicate wells were set for each group of treatment. The cells were collected from each well every 24 hours and continuously counted by a blood cell counting plate for 3 times. Glycogen of the L-15 culture medium was mainly galactose, and energy was mainly provided for the cells through mitochondria in such culture environment.

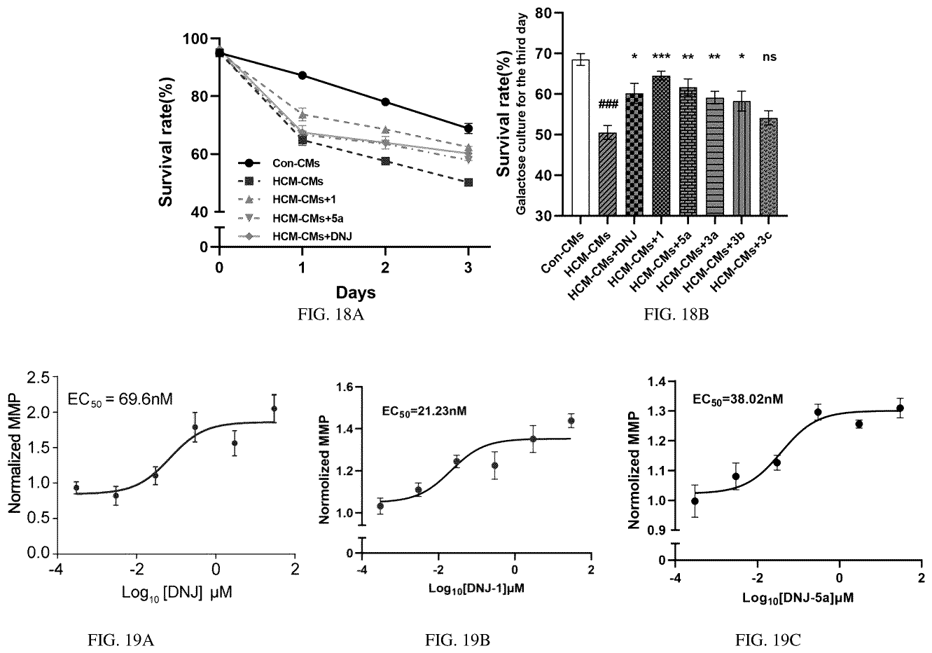

Results are shown in FIG. 18. DNJ-1 and DNJ-5a have optimal effects, which can significantly enhance the cell viability of HCM-iPSC-CMs with MT-RNR2 mutation in the galactose culture medium by improving mitochondrial functions, and have better performance compared with the lead compound. DNJ-3a, DNJ-3b and DNJ-3c also have certain degrees of enhancement effects.

### Example 6

HCM-iPSC-CMs with MT-RNR2 mutation were implemented with derivative treatments, including DNJ, DNJ-1 and DNJ-5a, at different concentrations, and a half effective concentration (EC50) was obtained by measuring the mitochondrial membrane potential.

The drug concentrations were diluted at a 10-fold gradient, and impacts on the mitochondrial membrane potential were observed under DNJ-1 treatment at different concentrations (0.0003, 0.003, 0.03, 0.3, 3, 30 M). A method for determining the mitochondrial membrane potential was the same as that previously described in Example 4.

Results are shown in FIG. 19. The half effective concentration of DNJ is 69.6 nM, the half effective concentration of DNJ-1 is 21.2 nM, and the half effective concentration of DNJ-5a is 38.02 nM, indicating that a maximum effect of 50% can be achieved at the concentrations. It is indicated that compared with the lead compound DNJ, DNJ-1 and DNJ-5a exert effects more rapidly and have higher drug activity.

### Example 7

An OPA1-EGFP protein was subjected to eukaryotic purification, and an interaction between the target protein and a small molecule compound was detected by microscale thermophoresis (MST) to obtain an affinity parameter, namely a K_{D} value, of the two.

A full-length sequence of OPA1 (NM_015560.2) was obtained by PCR from cDNA derived from HEK293T and then subjected to homologous recombination into a pcDNA3.1-Flag/His-EGFP empty vector to successfully construct a pcDNA3.1-OPA1-Flag/His-EGFP eukaryotic expression vector.

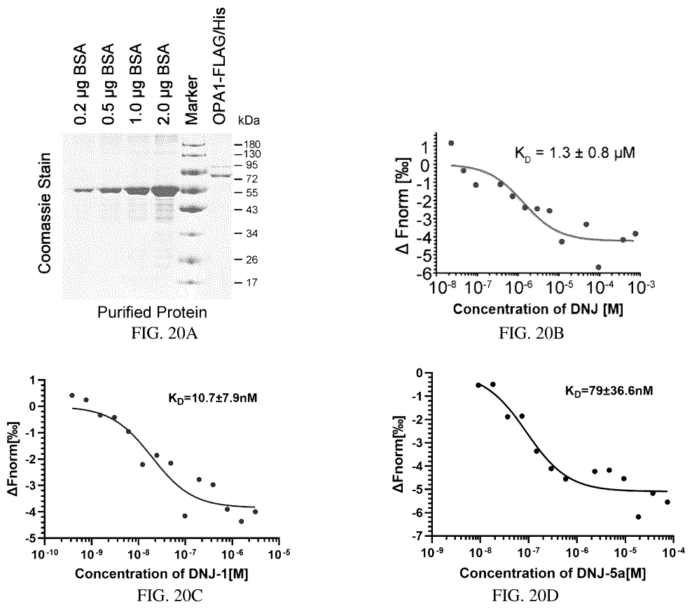

A Flag/His-OPA1-EGFP protein was overexpressed in HEK293T. After transfection for 48 hours, cells were collected and lysed. A protein supernatant was transferred to a new 15 mL centrifuge tube, Flag-beads were added, and incubation was performed on a turntable at 4° C. for 6-8 hours. Then, the supernatant was discarded with a magnetic grate. A lysis buffer was added and transferred to a new 1.5 mL EP tube, incubation was performed on a turntable at 4° C. for 3 min, and then a supernatant was sucked and discarded, wherein the step was repeated for 2 times. PBS and 3×Flag peptide were added, and incubation was performed overnight on a turntable at 4° C. After the incubation was completed, a liquid was transferred to a new EP tube by a magnetic grate. The liquid was the purified target protein. The purified protein was identified and quantified through electrophoresis gel running and a Coomassie brilliant blue staining experiment (FIG. 20A).

The derivative DNJ-1 (mother solution at 30 mM/L) was diluted 150-fold with Binding Buffer to serve as an initial concentration of tube 1. 16 PCR small tubes were prepared, and the above DNJ-1 was diluted by factors. 10 μL of Binding Buffer was added into the tubes 2-16. 20 μL of the diluted DNJ-1 was sucked and added into the PCR tube 1 for even mixing by blowing and beating with a pipette tip, and 10 μL of the liquid was sucked from the tube 1 and added into the tube 2. After the mixture was evenly mixed, 10 μL of the mixture was added into the tube 3, et cetera. 10 μL of the 40 nM purified EGFP-OPA1 mixture was added into each tube and analyzed by a Monolith NT.115 instrument. The Kd value was calculated.

The microscale thermophoresis (MST) is one of classic means used for detecting an interaction between a protein and a small molecule compound. According to the MST based on a thermophoresis effect, the affinity between a ligand and a target protein is detected by observing changes brought by a conformation or a ligand proximity effect after the ligand binds to the target protein. Results are shown in FIG. 20. Compared with the lead compound, the Kd values of DNJ-1 and DNJ-5a are significantly decreased, indicating that DNJ-1 and DNJ-5a have better targetability.

### Example 8

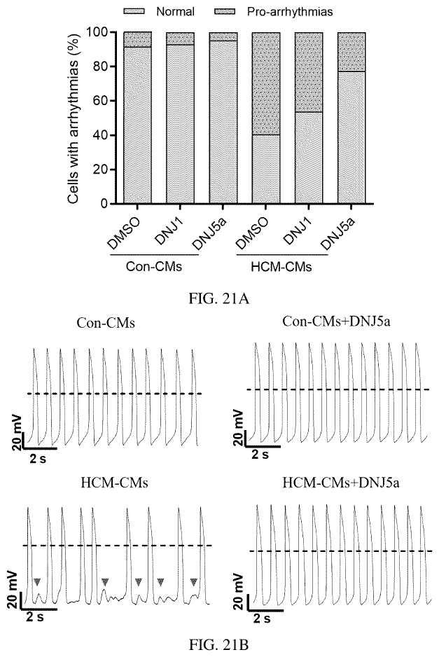

TrypLE was added into induced cardiomyocytes, and after digestion at 37° C. for 3 min, an RPMI complete culture medium was added. The cells were centrifuged at a low speed, resuspended with RPMI/B27, and inoculated into an 8 mm Matrigel-coated slide at a ratio of 1:10. The cells were placed in an environment with 5% of CO₂ for culture at 37° C. At 16 hours after the inoculation, the culture medium was replaced with a fresh RPMI/B27 culture medium, 30 μM small molecule drugs were added in an experimental group, and DMSO of an equal volume was added in a control group. At 48 hours after the culture, the action potential of the cardiomyocytes was recorded. Cell slides were removed, placed in a constant-temperature patch clamp bath at 37° C., and continuously perfused and rinsed with an extracellular fluid. Cell membranes of the cardiomyocytes were sucked and broken at a negative pressure, and the action potential of the cells capable of spontaneously pulsating was recorded. Data were collected and analyzed through Patch Master (HEKA), Fit Master (HEKA), Igor Pro (Wavemetrics) and Origin 6.1 (Microcal) software. Results are shown in FIG. 21. Compared with DNJ-1, DNJ-5a can improve the proportion of electrophysiologically abnormal cells in HCM-iPSC-CMs more significantly.

### Example 9

Model construction was performed by adopting C57BL/6J wild-type mice with subcutaneous implantation and perfusion of angiotensin II (AngII)/normal saline (Saline) through an Alzet Osmotic Pump 2004. Relevant statistical results were obtained after continuous feeding, modeling and administration for 4 weeks.

Model construction: Weighing and anesthetization were performed, and skin preparation and disinfection were performed in surgical areas. A 1 cm incision above a scapula of a front leg was made by using surgical scissors, wherein the incision was perpendicular to a tail part. The skin was cut carefully without damaging underlying tissues, a subcutaneous tunnel was made under the skin by using hemostatic forceps, the pump with a regulator head was completely pushed into a pocket (pointing toward a tail end of each mouse), and the incision was sutured.

Administration method: Intraperitoneal injection was adopted at a drug dose of 10 mg/kg/d, drugs were dissolved in a phosphate buffer solution (PBS) and administered twice a day in the morning and the evening, and a placebo group was given a phosphate buffer solution (PBS) of a same volume.

Mouse echocardiography: The mice were subjected to cardiac uhrasonography at 4 weeks after the modeling and administration. In brief, (1) skin preparation: hair was removed from chests below the xiphoid to left armpits of the mice by using a hair removal cream before ultrasonic detection; and (2) anesthetization: the mice were anesthetized by inhaling isoflurane, a flow rate of the isoflurane was rapidly decreased when the mice were in a light anesthesia state, then the mice were placed on a constant-temperature workbench at 37° C., four limbs of the mice were fixed with an adhesive tape until heart rates of the mice were controlled at about 480-540 beat/min, and at this time, ultrasonic detection was performed when the mice with the light anesthesia and the increased heart rates were capable of cooperating with ultrasonic operations.

In the experiment, a Vinno 6LAB high-resolution ultrasonic detector for small animals was used for detection in a two-dimensional (2D) parasternal short-axis M mode. Under image guidance, the position of an ultrasonic probe was rotated and adjusted until clearer short-axis images of left ventricles were obtained, and the images were acquired. Each indicator was detected for 3-6 cardiac cycles, and a mean value of the measured cardiac cycles was calculated.

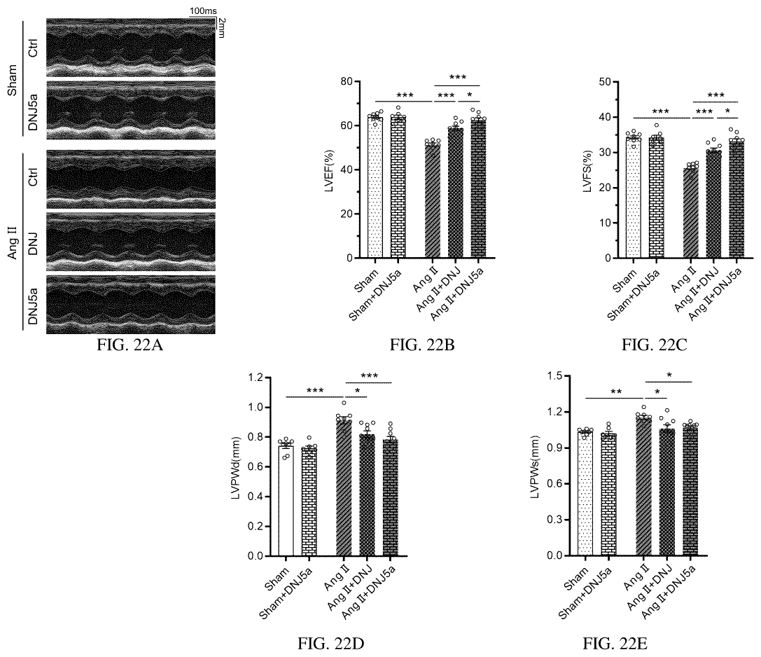

Statistical results of several key parameters of the cardiac uhrasonography are shown in FIG. 22. Compared with a control group, some key indicators in a modeling group, such as ejection fraction (EF) and fractional shortening (FS), are decreased significantly, and left ventricle posterior wall thickness during diastole (LVPWd) and left ventricle posterior wall thickness during systole (LVPWs) are increased significantly. In an administration group, relevant pathological phenotypes can be improved significantly after intervention. Moreover, compared with the lead drug DNJ, DNJ-5a has better performance, indicating that DNJ-5a has a better in vivo effect than the lead drug DNJ.

### Example 10

After continuous feeding, modeling and administration for 4 weeks, indicators, such as body weight, of mice were measured before sampling, then the mice were deeply anesthetized with isoflurane, and the mice were sacrificed after completely losing the sense of pain. Hearts were obtained after the mice were sacrificed, and indicators, such as heart weight, were measured.

In histological analysis, the hearts of the mice were collected, cut along maximum cross sections, and fixed with 4% paraformaldehyde. Subsequently, paraffin embedding, sectioning and hematoxylin-eosin (HE) staining of the maximum cross sections were performed. Steps are as follows: 1) treatment with xylene (I) for 9 min; 2) treatment with xylene (II) for 9 min; 3) treatment with xylene (III) for 9 min; 4) treatment with anhydrous ethanol I for 5 min and anhydrous ethanol II for 5 min; 5) treatment with 85% ethanol for 5 min; 6) treatment with 85% ethanol for 5 min; 7) rinsing with distilled water for 4-5 min for about 2-3 times; 8) staining with hematoxylin for 5 min; 9) rinsing slightly with running water to remove the hematoxylin solution for 1-3 seconds; 10) differentiation with 1% ethanol hydrochloride for 3 seconds; 11) rinsing with double distilled water for 10-30 seconds; 12) rinsing with PBS for 1 to 2 seconds; 13) staining with a 0.5% eosin solution for 2 min; 14) rinsing with distilled water for 2 seconds; 15) differentiation with 85% ethanol for 4 seconds; 16) treatment with 95% alcohol (I) for 2 min; 17) treatment with 95% alcohol (II) for 3 min; 18) treatment with anhydrous ethanol for 11 min; 19) treatment with carboxylol for about 8-9 min; 20) treatment with xylene (I) for 4 min; 21) treatment with xylene (II) for 4 min; 22) treatment with xylene (III) for 4.5 min; and 23) finally, sealing of sections with neutral gum for long-term storage. The sections were scanned by a scanner (3DHISTECH), and obtained and measured through software slideviewer.

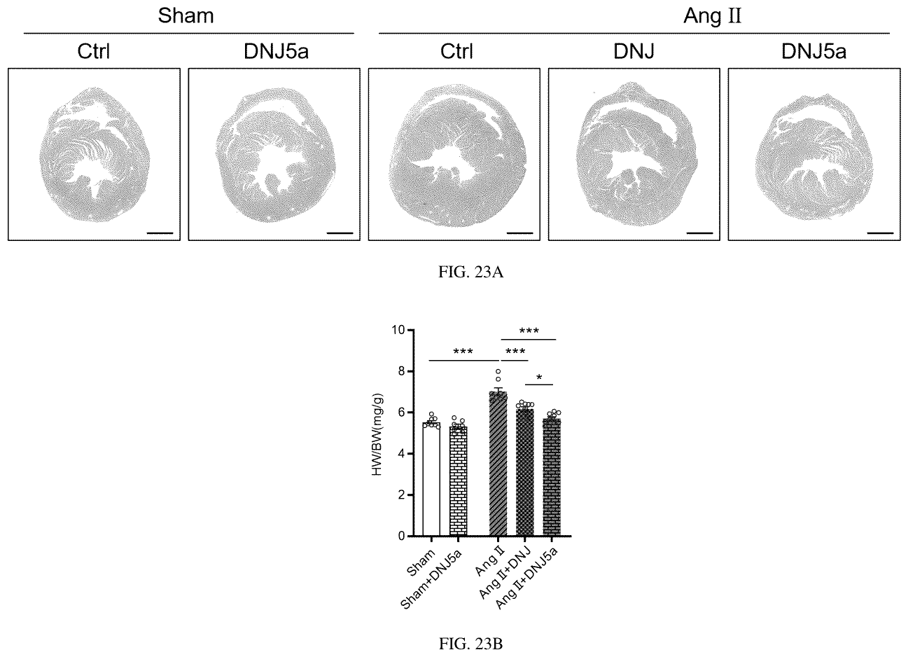

Results are shown in FIG. 23. Compared with a control group, the heart weight/body weight in a modeling group is significantly increased, and the ratio can be significantly decreased after drug intervention. Compared with the lead drug DNJ, DNJ-5a has better performance.

### Example 11

After mice were sacrificed, a small portion of left ventricular tissues were taken, and cardiac muscles were trimmed to achieve cubes with a size of 1 mm³. According to arrangement directions of mitochondria in the cardiac muscle tissues, tissues were selected respectively along long axes and short axes of hearts, and samples for transmission electron microscopy were prepared subsequently.

Steps are as follows: 1) fixation: the selected cardiac muscle tissue specimens were placed in a PBS buffer solution containing 2.5% of glutaraldehyde for fixation at room temperature for 2 hours and then for fixation at 4° C. overnight; 2) the specimens were rinsed with about 1 ml of 0.1 M PBS for 10 min for 3 times; 3) the specimens were fixed with about 50-100 μl (covering the samples) of 1% osmium acid for 1 hour; 4) the specimens were rinsed with water for 10 min for 3 times; 5) the specimens were fixed/stained with about 100 μl of a 2% uranyl acetate aqueous solution for 30 min; 6) dehydration: the specimens were respectively dehydrated with 50%, 70% and 90% ethanol for 15 min, 100% ethanol for 20 min, and 100% acetone for 20 min for 2 times; 7) penetration: the specimens were penetrated with an embedding agent and pure acetone (1:1) at room temperature for 2 hours and penetrated with an embedding agent and pure acetone (at a volume ratio of 3:1) overnight; and 8) embedding: the specimens were subjected to liquid change with a pure embedding agent, embedded in a correct direction, and placed at 30-37° C.

After polymerization, ultrathin sections and staining were entrusted to the Cryo-Electron Microscopy Center of Zhejiang University. Subsequently, the mitochondria of the tissues were photographed by a Talos 120 KV cryo-transmission electron microscope to obtain results.

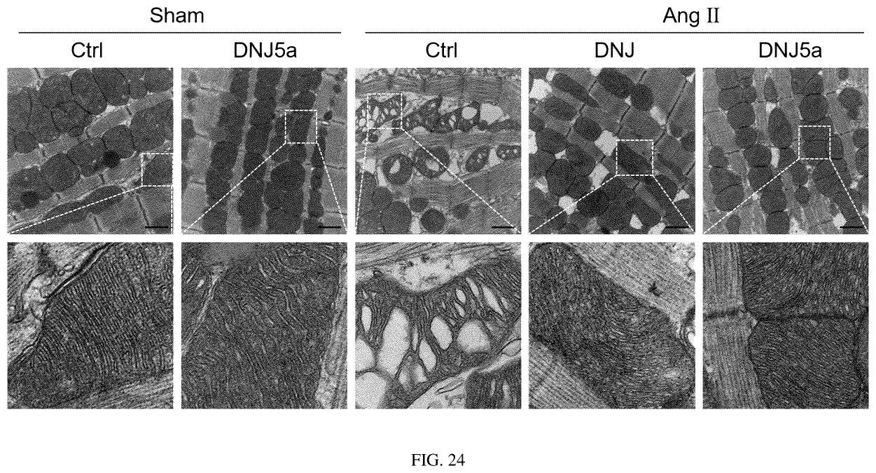

As shown in FIG. 24, compared with a control group, the mice in a modeling group have an obvious degree of mitochondrial damage in the hearts, swollen and vacuolated mitochondrial crista and disordered arrangement of muscle filaments, and the mice in an administration group can significantly improve a mitochondrial ultrastructure. Compared with the lead drug DNJ, DNJ-5a can more significantly improve the ultrastructure of the mitochondrial crista and decrease a vacuolation proportion.

1. A 1-deoxynojirimycin derivative, being one of the following:
(1) DNJ-1, having a structure of formula II: 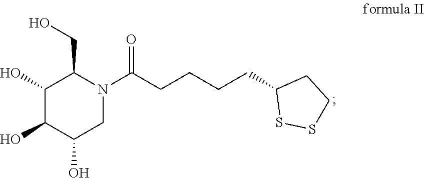 [SMILES: O=C(CCCC[C@@H]1CCSS1)N1C[C@H](O)[C@@H](O)[C@H](O)[C@H]1CO ; ";"]
(2) DNJ-5a, having a structure of formula VII: 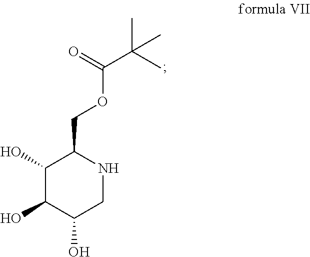 [SMILES: CC(C)(C)C(=O)OC[C@H]1NC[C@H](O)[C@@H](O)[C@@H]1O ; ";"]
(3) DNJ-5c, having a structure of formula VIII: 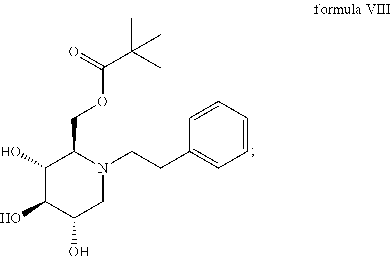 [SMILES: CC(C)(C)C(=O)OC[C@@H]1[C@@H](O)[C@H](O)[C@@H](O)CN1CCc1ccccc1 ; ";"]
and
(4) having a structure of general formula I-I: 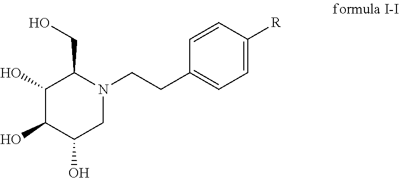
wherein R is selected from —H, —NO₂, and —X, wherein X is F, Cl, Br, or I.

2. The 1-deoxynojirimycin derivative according to claim 1, wherein R is selected as —X, and X is F.

3. A method of preparing an OPA1 agonist comprising the step of utilizing the 1-deoxynojirimycin derivative according to claim 1 or a pharmaceutically acceptable salt thereof.

4. A method for treating a disease that is related to unbalanced formation of an OPA1 multimer comprising the step of administering to a subject a drug comprising a 1-deoxynojirimycin derivative according to claim 1 or a pharmaceutically acceptable salt thereof.

5. The method according to claim 4, wherein the disease that is related to unbalanced formation of the OPA1 multimer is hypertrophic cardiomyopathy, epicophosis, optic atrophy, progressive external ophthalmoplegia and ataxia, progressive myoclonic epilepsy, spastic paresis and intestinal motility disorder, or retinal degeneration.

6. The method according to claim 5, wherein the disease that is related to unbalanced formation of the OPA1 multimer is caused by mitochondrial dysfunction.

7. A drug for treating a disease that is related to unbalanced formation of an OPA1 multimer, wherein an effective ingredient is the 1-deoxynojirimycin derivative according to claim 1 or a pharmaceutically acceptable salt thereof.

8. The drug according to claim 7, wherein the disease that is related to unbalanced formation of the OPA1 multimer is hypertrophic cardiomyopathy, epicophosis, optic atrophy, progressive external ophthalmoplegia and ataxia, progressive myoclonic epilepsy, spastic paresis and intestinal motility disorder, or retinal degeneration.

9. The drug according to claim 8, wherein the hypertrophic cardiomyopathy, epicophosis, optic atrophy, progressive external ophthalmoplegia and ataxia, progressive myoclonic epilepsy, spastic paresis and intestinal motility disorder, or retinal degeneration is caused by mitochondrial dysfunction.

10. A method for preparing the 1-deoxynojirimycin derivative according to claim 1, wherein
(i) when the 1-deoxynojirimycin derivative is the (1), compounds a and b1 are taken and allowed to undergo a condensation reaction in a solvent to obtain the corresponding 1-deoxynojirimycin derivative, wherein the compound a is 1-deoxynojirimycin, and the compound b1 is: 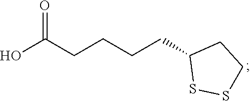 [SMILES: O=C(O)CCCC[C@@H]1CCSS1 ; ";"]
(ii) when the 1-deoxynojirimycin derivative is the (2), 1-deoxynojirimycin first reacts with benzyl chloroformate to generate an intermediate product 1: 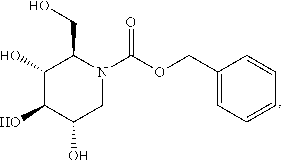 [SMILES: O=C(OCc1ccccc1)N1C[C@H](O)[C@@H](O)[C@H](O)[C@H]1CO ; ","]
then, the intermediate product 1 reacts with PivCl to obtain an intermediate product 2: 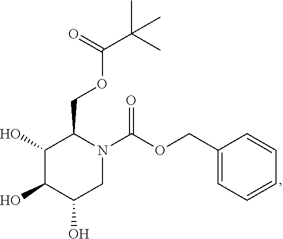 [SMILES: CC(C)(C)C(=O)OC[C@@H]1[C@@H](O)[C@H](O)[C@@H](O)CN1C(=O)OCc1ccccc1 ; ","]
and finally, the intermediate product 2 is reduced to obtain the 1-deoxynojirimycin derivative;
(iii) when the 1-deoxynojirimycin derivative is the (3), 1-deoxynojirimycin first reacts with 2-bromoethylbenzene to generate an intermediate product: 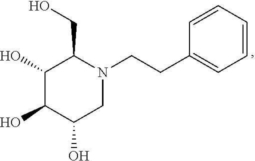 [SMILES: OC[C@@H]1[C@@H](O)[C@H](O)[C@@H](O)CN1CCc1ccccc1 ; ","]
and then, the intermediate product reacts with PivCl to obtain the 1-deoxynojirimycin derivative; and
(iv) when the 1-deoxynojirimycin derivative is the (4), compounds a and b4 are taken and allowed to undergo a condensation reaction in a solvent to obtain the corresponding 1-deoxynojirimycin derivative, wherein the compound a is 1-deoxynojirimycin, and the compound b4 is: [chemical structure]
